(19) United States
(12) Patent Application Publication
(10) Pub. No.: US 2017/0296626 A1
(43) Pub. Date: Oct. 19, 2017

(54) EXOSOME ISOLATION

(71) Applicant: EXERKINE CORPORATION, Hamilton (CA)

(72) Inventor: Mark TARNOPOLSKY, Hamilton (CA)

(21) Appl. No.: 15/449,489

(22) Filed: Mar. 3, 2017

Related U.S. Application Data
(63) Continuation-in-part of application No. PCT/CA2015/050853, filed on Sep. 4, 2015.
(60) Provisional application No. 62/211,312, filed on Aug. 28, 2015.
(60) Provisional application No. 62/112,940, filed on Feb. 6, 2015.
(60) Provisional application No. 62/046,518, filed on Sep. 5, 2014.

(51) Int. Cl. A61K 38/18; A61K 9/51

(52) U.S. Cl. CPC A61K 38/1866; A61K 9/5176; A61K 9/5192

(57) ABSTRACT

Methods of isolating exosomes from a biological sample is provided. In one embodiment, the method may include a series of optional centrifugation steps, and comprises exosome precipitation using a PEG-based solution followed by resuspension in a saccharide-based solution such as trehalose. The method advantageously results in essentially pure exosomes that maintain integrity and stability. The exosomes are useful for the in vivo delivery of cargo, including macromolecules such as protein and nucleic acid.

### FIELD OF THE INVENTION

The present invention generally relates to exosomes, and more particularly relates to a method of isolating exosomes from a biological sample.

### BACKGROUND OF THE INVENTION

In organisms, tissues and cells must continuously correspond with each other to best adapt to their surrounding microenvironment. To date, the transmission of signals between cells and tissues has been described by protein-based signaling systems exemplified by enzymes, hormones, cytokines, and chemokines. However, there are a plethora of peptides that cannot survive the exposed circulatory environment. Additionally, both mRNA and miRNA are extremely labile in the extracellular environment and require an encapsulated venue for transfer between tissues and organs. Thus, these factors are secreted in small double-membrane extracellular vesicles (ECVs) including exosomes, microvesicles, and apoptotic bodies. Depending on their cellular site of origin, these vesicles have distinct structural and biochemical properties that affect their function and role in biological systems. For example, exosomes are generally homogeneous and are about 40-120 nm in size, while microvesicles and apoptotic bodies are heterogeneous in appearance and from 100 nm to 1000 nm and greater than 1000 nm in size, respectively. Together, these ECVs contain a variety of bioactive molecules, including proteins, biolipids, and nucleic acids, which can be transferred between cells without direct cell-to-cell contact. Consequently, ECVs represent a form of intercellular communication, which could play a role in both physiological and pathological processes. Growing evidence indicates that circulating ECVs contribute to the development of cancer, inflammation, and autoimmune and cardiovascular diseases.

Exosome signaling and biology has been extensively studied in the last decade inrelation to their role in various pathologies and disease biomarker discovery. However, there are a few studies deciphering the physiological role of exosomes in cellular metabolism and promoting inter-organ cross-talk. One of the major limitations in studying exosomes is lack of a standardized/functional exosome isolation protocol. Many of the isolation protocols that are published lack the capacity to yield a purified exosome population of sufficient quantity and quality for biochemical analyses and various other downstream applications, including the delivery of therapeutic payloads (protein, mRNA, miRNA, etc.). Additionally, the non-ultracentrifuge/filtration-based methodologies commercially available in the form of kits have severe shortcomings in their ability to isolate a pure exosomal fraction and/or to isolate exosomes of sufficient quality for biological evaluation or therapeutic delivery.

It would be desirable, thus, to provide an improved method for isolating exosomes from a biological sample for research purposes, and/or to develop exosomes for use in diagnostics or therapeutics.

### SUMMARY OF THE INVENTION

A novel exosome isolation method has now been developed as described herein.

Accordingly, in a first aspect of the present invention, a method of isolating exosomes from a biological sample is provided comprising the steps of:

i) exposing the biological sample to an optional first centrifugation step to remove cellular debris greater than about 7-10 microns in size from the sample and obtaining the supernatant following centrifugation;

ii) subjecting the supernatant from step i) to an optional centrifugation step to remove microvesicles therefrom;

iii) optionally microfiltering the supernatant from step ii) and collecting the microfiltered supernatant;

iv) combining the microfiltered supernatant from step iii) with a polyethylene glycol solution to precipitate the exosomes and subjecting the solution to at least one round of ultracentrifugation to obtain an exosome pellet; and

v) re-suspending the exosome pellet from step iv) in a trehalose solution and conducting an optionalcentrifugation step to remove vesicles having a diameter of greater than 140 nm from the solution.

In another aspect of the invention, a method of isolating exosomes from a biological sample is provided comprising the steps of:

i) exposing the biological sample to a first centrifugation to remove cellular debris greater than about 7-10 microns in size from the sample and obtaining the supernatant following centrifugation;

ii) subjecting the supernatant from step i) to centrifugation to remove microvesicles therefrom;

iii) microfiltering the supernatant from step ii) and collecting the microfiltered supernatant;

iv) subjecting the microfiltered supernatant from step iii) to at least one round of ultracentrifugation to obtain an exosome pellet;

v) re-suspending the exosome pellet from step iv) in a physiological solution, conducting a second ultracentrifugation in a density gradient and removing the exosome pellet fraction therefrom.

In another aspect of the invention, an exosome pellet or solution is provided comprising exosomes essentially free from particles having a diameter less than 20 nm or greater than 140 nm.

In another aspect of the invention, a composition is provided comprising exosomes essentially free from particles having a diameter greater than or less than 20-140 nm, wherein said exosomes are loaded with exogenous cargo.

In another aspect, a method of in vivo delivery of exogenous cargo to a mammal is provided comprising the step of administering to the mammal a physiologically acceptable exosome solution which is essentially free from particles having a diameter less than 20 nm or greater than 140 nm, wherein the exosomes are loaded with the exogenous cargo.

In a further aspect of the present invention, a kit is provided, useful to conduct a method of isolating exosomes from a biological sample as herein described.

These and other aspects of the present invention will become apparent in the detailed description that follows, by reference to the following figures.

### BRIEF DESCRIPTION OF THE FIGURES

FIG. 1 graphically illustrates the size of exosomes from a human (A) and mouse (B) sample using a method according to an embodiment of the invention;

FIG. 2 graphically illustrates the size of exosomes isolated from corn husks (A) and pomegranate seeds (B);

FIG. 3 graphically illustrates the size (A) and zeta potential (B) of exosomes as an embodiment of the present isolation method is scaled up;

FIG. 4 graphically illustrates the exosome protein yield of an embodiment of the present isolation method is scaled up;

FIG. 5 illustrates electron microscopy analyses of products isolated using commercial exosome isolation kits (A) and the exosome product using an isolation method according to an aspect of the invention (B);

FIG. 6 illustrates the exosome concentration achieved using an isolation method according to one aspect of the present invention (EX1-6) in comparison to exosome concentrations achieved using commercial isolation kits (S1-6) and BSA standards both colorimetrically (A) and graphically (B);

FIG. 7 graphically illustrates the size of exosomes (diameter in nm) isolated from a sample using alternative exosome isolation methods (AB);

FIG. 8 graphically compares GADPH expression levels in a sample when GADPH siRNA is co-delivered with a transfection agent and delivered by exosomes isolated according to an alternative prior method;

FIG. 9 graphically compares GADPH expression levels in a sample when GADPH siRNA is co-delivered with a transfection agent and delivered by exosomes isolated according to an embodiment of the invention;

FIG. 10 graphically compares the uptake of siRNA, miRNA, mRNA and peptide by exosomes isolated according to an embodiment of the invention and an alternative prior isolation method;

FIG. 11 graphically compares the survival of mice treated with exosomes isolated according to an embodiment of the invention and exosomes isolated by an alternative prior isolation method;

FIG. 12 graphically compares expression levels of VEGF-a delivered to cardiomyocytes cardiomyocytes as modRNA-Vegfa (A), modRNA-Vegfa-loaded exosomes (AIB) or mRNA-Vegfa-loaded exosomes (B);

FIG. 13 graphically compares the bio distribution of exosomes over time;

FIG. 14 graphically compares the biological activity of SED and END exosomes isolated using a method according to an aspect of the present invention to the activity of the equivalent exosomes isolated using a commercially available kit;

FIG. 15 graphically compares the results of activity endurance testing of control SED and END mice to that of sedentary (SED) mice treated with SED and END exosomes isolated using a method according to an aspect of the present invention;

FIG. 16 illustrates electron microscopy analyses of exosomes isolated using a polyethylene glycol (PEG)-based method from various sources including: human serum (A), rat serum (B), mouse serum (C), human plasma (D), rat plasma (E), mouse plasma (F) and bovine milk (G);

FIG. 17 graphically illustrates the size of exosomes from human serum (A), mouse serum (B), rat serum (C) and bovine milk (D) samples using a PEG-based exosome isolation method;

FIG. 18 illustrates a Western Blot analyses of exosomes isolated using a PEG-based exosome isolation method from mouse plasma and serum sources (A) and graphically illustrates Ponceau staining of mouse serum exosomes isolated using a PEG-based isolation method (in pellet and supernatant) as compared to exosomes isolated using a commercially available kit;

FIG. 19 graphically illustrates expression levels of luciferase delivered in vivo to the liver (A), lung (B) and spleen (C) tissues using exosomes isolated with a PEG-based method and loaded with exogenous luciferase mRNA;

FIG. 20 graphically illustrates expression levels of luciferase delivered in vivo to the liver (A), lung (B), spleen (C) and brain (D) tissues using exosomes isolated with a PEG-based method and loaded with exogenous recombinant luciferase protein; and

FIG. 21 graphically illustrates fluorescently labeled exosome uptake into liver tissue in vivo using exosomes isolated with a PEG-based method.

### DETAILED DESCRIPTION OF THE INVENTION

A novel method of isolating exosomes from a biological sample is provided. The method includes the steps of: i) optionally exposing the biological sample to a first centrifugation to remove cellular debris greater than about 7-10 microns in size from the sample and obtaining the supernatant following centrifugation; ii) optionally subjecting the supernatant from step i) to centrifugation to remove microvesicles and apoptotic bodies therefrom; iii) optionally microfiltering the supernatant from step ii) and collecting the microfiltered supernatant; iv) combining the microfiltered supernatant from step iii) with a polyethylene glycol solution to precipitate the exosomes and subjecting the solution to at least one round of centrifugation to obtain an exosome pellet; and v) re-suspending the exosome pellet from step iv) in a trehalose solution and conducting an optional centrifugation step to remove vesicles having a diameter of greater than 140 nm from the solution.

The term “exosome” refers to cell-derived vesicles having a diameter of between about 20-140 nm, such as between 40 and 120 nm, preferably a diameter of about 50-100 nm, for example, a diameter of about 60 nm, 70 mu, 80 nm, 90 nm, or 100 mm Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g. immature dendritic cells (wild-type or immortalized), induced and non-induced pluripotent stem cells, fibroblasts, platelets, immune cells, reticulocytes, tumour cells, mesenchymal stem cells, satellite cells, hematopoietic stem cells, pancreatic stem cells, white and beige pre-adipocytes and the like). As one of skill in the art will appreciate, cultured cell samples will be in the cell-appropriate culture media (using exosome-free serum). Exosomes include specific surface markers not present in other vesicles, including surface markers such as tetraspanins, e.g. CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein). Exosomes may also be obtained from a non-mammal or from cultured non-mammalian cells. As the molecular machinery involved in exosome biogenesis is believed to be evolutionarily conserved, exosomes from non-mammalian sources include surface markers which are isoforms of mammalian surface markers, such as isoforms of CD9 and CD63, which distinguish them from other cellular vesicles. As used herein, the tem “mammal” is meant to encompass, without limitation, humans, domestic animals such as dogs, cats, horses, cattle, swine, sheep, goats and the like, as well as non-domesticated animals such as, but not limited to, mice, rats and rabbits. The term “non-mammal” is meant to encompass, for example, exosomes from microorganisms such as bacteria, flies, worms, plants, fruit/vegetables (e.g. corn, pomegranate) and yeast.

In accordance with an aspect of the present invention, the process of isolating exosomes from a biological sample includes a first optional step of removing undesired large cellular debris from the sample, i.e. cells, cell components, apoptotic bodies and the like greater than about 7-10 microns in size. This first step is generally conducted by centrifugation, for example, at 1000-4000×g for 10 to 60 minutes at 4 ° C., preferably at 1500-2500×g, e.g. 2000×g, for a selected period of time such as 10-30 minutes, 12-28 minutes, 14-24 minutes, 15-20 minutes or 16, 17, 18 or 19 minutes. As one of skill in the art will appreciate, a suitable commercially available laboratory centrifuge, e.g. Thermo-Scientific™ or Cole-Parmer™, is employed to conduct this isolation step. To enhance exosome isolation, the resulting supernatant is subjected to an additional optional centrifugation step to further remove cellular debris and apoptotic bodies, such as debris that is at least about 7-10 microns in size, by repeating this first step of the process, i.e. centrifugation at 1000-4000×g for 10 to 60 minutes at 4° C., preferably at 1500-2500×g, e.g. 2000×g, for the selected period of time.

Following removal of cell debris, the supernatant resulting from the first

The supernatant is then optionally filtered to remove debris, such as bacteria and larger microvesicles, having a size of about 0.22 microns or greater, e.g. using microfiltration. The filtration may be conducted by one or more passes through filters of the same size, for example, a 0.22 micron filter. Alternatively, filtration using 2 or more filters may be conducted, using filters of the same or of decreasing sizes, e.g. one or more passes through a 40-50 micron filter, one or more passes through a 20-30 micron filter, one or more passes through a 10-20 micron filter, one or more passes through a 0.22-10 micron filter, etc. Suitable filters for use in this step include the use of 0.45 and 0.22 micron filters.

The microfiltered supernatant (filtrate) may then be combined with a polyethylene glycol (PEG) solution to precipitate exosomes within the filtrate. As would be appreciated by one of skill in the art, a variety of PEG formulations may be used. Preferably, these formulations comprise PEG chain lengths having an average molecular weight of between about 400 to 20,000 daltons (e.g. 1000 to 10,000 daltons, such as 6000 daltons). Similarly, the exosome-PEG solutions may have varying final concentrations of PEG, for example, a final concentration of PEG may be between about 5-15% (such as 8%). Preferably, the filtrate is combined with an equal volume of the PEG solution, having a strength in the range of about 10-20% PEG. Salts may be added to the PEG solution to enhance the precipitation of exosomes. Preferably, a salt such as NaCl is added to the PEG solution so that the final concentration of salt in the exosome-PEG-salt solution is between about 50 to 1,000 mM (such as 500 mM). The PEG-filtrate is gently mixed and incubated under conditions suitable for exosome precipitation, e.g. incubated for 30 minutes at 4° C. Some samples may require a longer incubation period for exosome precipitation to occur.

Following incubation, the precipitated exosomes were pelleted by centrifugation, e.g. at 10,000×g for 10 min at 4° C., and the pellet was solubilized in a suitable saccharide solution, such as a trehalose solution, that is effective to reduce aggregation of the exosomes. The saccharide is preferably solubilized in a physiological buffer, such as saline or PBS. In one embodiment, a trehalose solution of various concentrations is effective at reducing the aggregation of exosomes, such as a trehalose concentration between 10 mM to 1,000 mM (e.g. 500 mM).

To remove non-exosome extracellular vesicles (i.e. vesicles larger than 140 nm), the trehalose exosome solution may be subjected to further optional centrifugation or ultracentrifugation steps, for example, at 15,000×g-150,000×g for 1 hr at 4° C. If ultracentrifugation is performed, exosomes will be present in both the resultant pellet and supernatant fractions, generally with a larger quantity of exosomes in the supernatant.

To enhance removal of impurities that are smaller than the exosomes, e.g. smaller than 20 nm, the exosome-trehalose solution may be subjected to an optional ultrafiltration step using either a direct-flow filtration technique (such as a centrifugal spin filter) or a cross-flow filtration technique (such as a tangential flow system). As would be appreciated by one of skill in the art, filtration membranes suitable for this step may possess a molecular weight cut-off (MWCO) rating in the range of 3-500 kDa and preferably between 100-300 kDa.

In another embodiment, exosome isolation may include the steps of: i) exposing the biological sample to a first centrifugation to remove cellular debris greater than about 7-10 microns in size from the sample and obtaining the supernatant following centrifugation; ii) subjecting the supernatant from step i) to centrifugation to remove microvesicles and apoptotic bodies therefrom; iii) microfiltering the supernatant from step ii) and collecting the microfiltered supernatant; iv) subjecting the microfiltered supernatant from step iii) to at least one round of ultracentrifugation to obtain an exosome pellet; and v) re-suspending the exosome pellet from step iv) in a physiological solution and conducting a second ultracentrifugation in a density gradient and remove the exosome pellet fraction therefrom.

The centrifugation and filtration steps (steps i)-iii)) are as previously described.

Following the initial centrifugation and filtration steps, the exosomal solution is then subjected to ultracentrifugation to pellet exosomes and any remaining contaminating microvesicles (between 100-220 nm). This ultracentrifugation step is conducted at 110,000-170,000×g for 1-3 hours at 4° C., for example, 170,000×g for 3 hours. This ultracentrifugation step may optionally be repeated, e.g. 2 or more times, in order to enhance results. Any commercially available ultracentrifuge, e.g. Thermo-Scientific™ or Beckman™, may be employed to conduct this step. The exosome-containing pellet is removed from the supernatant using established techniques and re-suspended in a suitable physiological solution.

Following ultracentrifugation, the re-suspended exosome-containing pellet is subjected to density gradient separation to separate contaminating microvesicles from exosomes based on their density. Various density gradients may be used, including, for example, a sucrose gradient, a colloidal silica density gradient, an iodixanol gradient, or any other density gradient sufficient to separate exosomes from contaminating microvesicles (e.g. a density gradient that functions similar to the 1.100-1.200 g/ml sucrose fraction of a sucrose gradient). Thus, examples of density gradients include the use of a 0.25-2.5 M continuous sucrose density gradient separation, e.g. sucrose cushion centrifugation, comprising 20-50% sucrose; a colloidal silica density gradient, e.g. Percoll™ gradient separation (colloidal silica particles of 15-30 nm diameter, e.g. 30%/70% w/w in water (free of RNase and DNase), which have been coated with polyvinylpyrrolidone (PVP)); and an iodixanol gradient, e.g. 6-18% iodixanol. The resuspended exosome solution is added to the selected gradient and subjected to ultracentrifugation at a speed between 110,000-170,000×g for 1-3 hours. The resulting exosome pellet is removed and re-suspended in physiological solution.

Depending on the density gradient used, the re-suspended exosome pellet resulting from the density gradient separation may be ready for use. For example, if the density gradient used is a sucrose gradient, the appropriate sucrose fractions are collected and may be combined with other collected sucrose fractions, and the resuspended exosome pellet is ready for use, or may preferably be subjected to an ultracentrifugation wash step at a speed of 110,000-170,000×g for 1-3 hours at 4° C. If the density gradient used is, for example, a colloidal silica (Percoll™) or a iodixanol density gradient, then the resuspended exosome pellet may be subjected to additional wash steps, e.g. subjected to one to three ultracentrifugation steps at a speed of 110,000-170,000× g for 1-3 hours each at 4° C., to yield an essentially pure exosome-containing pellet. The pellet is removed from the supernatant and may be re-suspended in a physiologically acceptable solution for use.

As one of skill in the art will appreciate, the exosome pellet from any of the centrifugation or ultracentrifugation steps may be washed between centrifugation steps using an appropriate physiological solution, e.g. sterile PBS, sterile 0.9% saline or sterile carbohydrate-containing 0.9% saline buffer.

The present methods advantageously provide a means to obtain mammalian and non-mammalian exosomes which are useful therapeutically. In some embodiments, the methods yield exosomes which exhibit a high degree of purity, for example, at least about 50% pure, and preferably, at least about 60%, 70%, 80%, 90% or 95% or greater pure. Preferably, the exosomes are “essentially free” from cellular debris, apoptotic bodies and microvesicles having a diameter less than 20 nm or greater than 140 nm, and preferably less than 40 nm or greater than 120 nm, and which are biologically intact, e.g. not clumped or in aggregate form, and not sheared, leaky or otherwise damaged. Exosomes isolated according to the methods described herein exhibit a degree of stability, that may be evidenced by the zeta potential of a mixture/solution of such exosomes, for example, a zeta potential of at least a magnitude of ±10 mV, e.g. ≦−10 or ≧+10, and preferably, a magnitude of at least 20 my, 30 mV, 40 mV, 50 mV, 60 mV, 70 mV, 80 mV, or greater. The term “zeta potential” refers to the electrokinetic potential of a colloidal dispersion, and the magnitude of the zeta potential indicates the degree of electrostatic repulsion between adjacent, similarly charged particles (exosomes) in a dispersion. For exosomes, generally the higher the magnitude of the zeta potential, the greater the stability of the exosomes.

Moreover, high quantities of exosomes are achievable by the present isolation method. With the PEG-based method, 1 mL of serum yields about 5-10 mg of protein. With the ultracentrifugation/density gradient method, 1 mL of serum or 15-20 mL of cell culture spent media (from at least about 2×10⁶ cells) yields about 100-2000 μg total protein. Thus, solutions comprising exosomes at a concentration of at least about 5 μg/μL, and preferably at least about 10-25 μg/μL, may readily be prepared due to the high exosome yields obtained by the present method. The term “about” as used herein with respect to any given value refers to a deviation from that value of up to 10%, either up to 10% greater, or up to 10% less.

Exosomes isolated in accordance with the methods herein described, beneficially retaining integrity, and exhibiting purity (being “essentially free” from entities having a diameter less than 20 nm and or greater than 140 nm), stability and biological activity both in vitro and in vivo, have not previously been achieved. Thus, the present exosomes are uniquely useful, for example, diagnostically and/or therapeutically. They have also been determined to be non-allergenic, and thus, safe for autologous, allogenic, and xenogenic use.

Exosomes obtained using the present method may be formulated for therapeutic use by combination with a pharmaceutically or physiologically acceptable carrier. The expressions “pharmaceutically acceptable” or “physiologically acceptable” means acceptable for use in the pharmaceutical and veterinary arts, i.e. not being unacceptably toxic or otherwise unsuitable for Physiological use. As one of skill in the art will appreciate, the selected carrier will vary with intended utility of the exosome formulation. In one embodiment, exosomes are formulated for administration by infusion or injection, e.g. subcutaneously, intraperitoneally, intramuscularly or intravenously, and thus, are formulated as a suspension in a medical-grade, physiologically acceptable carrier, such as an aqueous solution in sterile and pyrogen-free form, optionally, buffered or made isotonic. The carrier may be distilled water (DNase- and RNase-free), a sterile carbohydrate-containing solution (e.g. sucrose or dextrose) or a sterile saline solution comprising sodium chloride and optionally buffered. Suitable saline solutions may include varying concentrations of sodium chloride, for example, normal saline (0.9%), half-normal saline (0.45%), quarter-normal saline (0.22%), and solutions comprising greater amounts of sodium chloride (e.g. 3%-7%, or greater). Saline solutions may optionally include additional components, e.g. carbohydrates such as dextrose and the like. Examples of saline solutions including additional components, include Ringer's solution, e.g. lactated or acetated Ringer's solution, phosphate buffered saline (PBS), TRIS (hydroxymethyl) aminomethane hydroxymethyl) aminomethane)-buffered saline (TBS), Hank's balanced salt solution (HBSS), Earle's balanced solution (EBSS), standard saline citrate (SSC), HEPES-buffered saline (HBS) and Gey's balanced salt solution (GBSS).

In other embodiments, exosomes are formulated for administration by routes including, but not limited to, oral, intranasal, enteral, topical, sublingual, intra-arterial, intramedullary, intrathecal, inhalation, ocular, transdermal, vaginal or rectal routes, and will include appropriate carriers in each case. For example, exosome compositions for topical application may be prepared including appropriate carriers. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents, anti-oxidants and other preservatives may be added to the composition to prevent microbial growth and/or degradation over prolonged storage periods.

Alternatively, the exosome pellet may be stored for later use, for example, in cold storage at 4° C., in frozen form or in lyophilized form, prepared using well-established protocols. The exosome pellet may be stored in any physiological acceptable carrier, optionally including cryogenic stability and/or vitrification agents (e.g. DMSO, glycerol, trehalose, polyhydroxylated alcohols (e.g. methoxylated glycerol, propylene glycol), M22 and the like).

Exosomes isolated according to the present methods, in view of their unique properties (e.g. purity, integrity and stability) may advantageously be used as a vehicle for the delivery in vivo of cargo, e.g. exogenous cargo such as biomaterials, therapeutic compounds or other entities, in the treatment of disease or other conditions in mammals. Loading of the present isolated exosomes with exogenous cargo may be achieved due to the purity and stability of the present exosomes. Examples of cargo that may be delivered in vivo using the present exosomes include exogenous materials that do not exist naturally in exosomes (originate from an external source), such as, but not limited to, nucleic acid molecules such as DNA (both nuclear and mitochondrial), RNA such as mRNA, tRNA, miRNA, and siRNA, aptamers and other nucleic acid-containing molecules, peptides, proteins, ribozymes, carbohydrates, polymers, therapeutics, small molecules and the like. In one embodiment, the present isolated exosomes are particularly useful for the delivery of compounds having a secondary structure (e.g. miRNA, mRNA, protein/peptide), as well as macromolecules or large compounds, e.g. nucleic acid molecules which comprising more than 20 base pairs, e.g. more than 50 base pairs or more than 100 base pairs, peptides, proteins, and the like.

Cargo may be introduced into the present exosomes using methods established in the art for introduction of cargo into cells. Thus, cargo may be introduced into exosomes, for example, using electroporation applying voltages in the range of about 20-1000 V/cm. Transfection using cationic lipid-based transfection reagents may also be used to introduce cargo into exosomes. Examples of suitable transfection reagents include, but are not limited to, Lipofectamine® MessengerMAX™ Transfection Reagent, Lipofectamine® RNAiMAX Transfection Reagent, Lipofectamine® 3000 Transfection Reagent, or Lipofectamine® LTX Reagent with PLUS™ Reagent. For cargo loading, a suitable amount of transfection reagent is used and may vary with the reagent, the sample and the cargo. For example, using Lipofectamine® MessengerMAX™ Transfection Reagent, an amount in the range of about 0.15 uL to 10 uL may be used to load 100 ng to 2500 ng mRNA or protein into exosomes. Other methods may also be utilized to introduce cargo into exosomes, for example, the use of cell-penetrating peptides for protein introduction.

In view of the integrity and stability of the exosomes isolated according to the present invention, they advantageously permit loading of a desired cargo in an amount of at least about 1 ng mRNA or miRNA/10 ug of exosomal protein or 30 ug protein/10 ug of exosomal protein.

As will be appreciated by one of skill in the art, prior or subsequent to loading with cargo, the present exosomes may be further altered by inclusion of a targeting moiety to enhance the utility thereof as a vehicle for delivery of cargo. In this regard, exosomes may be engineered to incorporate an entity that specifically targets a particular cell to tissue type. This target-specific entity, e.g. peptide having affinity for a receptor or ligand on the target cell or tissue, may be integrated within the exosomal membrane, for example, by fusion to an exosomal membrane marker (as previously described) using methods well-established in the art.

The use of exosomes for the delivery in vivo of exogenous cargo loaded therein is also provided, along with related methods. A method of in vivo delivery of exogenous cargo to a mammal comprises the step of administering to the mammal a physiologically acceptable exosome solution which is essentially free from particles having a diameter less than 20 nm or greater than 140 nm. The exosomes are loaded with the exogenous cargo as above-described. The method is useful for the delivery of exogenous cargo to various tissues, including but not limited to, muscle, heart, brain, liver, kidney, lung, inguinal white adipose tissue, brown adipose tissue, pancreas or colon.

In another aspect of the invention, a kit is provided comprising one or more reagents or materials useful to conduct the present exosome isolation method, and instructions detailing how to conduct the method, e.g. instructions indicating the method comprises the steps of: i) optionally exposing the biological sample to a first centrifugation to remove cellular debris greater than about 7-10 microns in size from the sample and obtaining the supernatant following centrifugation; ii) optionally subjecting the supernatant from step i) to centrifugation to remove microvesicles and apoptotic bodies therefrom; iii) optionally microfiltering the supernatant from step ii) and collecting the microfiltered supernatant; iv) combining the microfiltered supernatant from step iii) with a polyethylene glycol solution to precipitate the exosomes and subjecting the solution to at least one round of centrifugation to obtain an exosome pellet; and v) re-suspending the exosome pellet from step iv) in a trehalose solution and conducting an optional centrifugation step to remove vesicles having a diameter of greater than 140 nm from the solution.

Thus, in addition to instructions, the kit may comprise solutions useful to conduct the method, such as one or more physiological solutions for re-suspending exosome-containing pellets following centrifugation of a sample, buffers, washing solutions, PEG solution and/or a trehalose solution. It may additionally include one or more materials such as biological sample containers, test tubes, centrifuge tubes, microfilters and the like.

Embodiments of the invention are described in the following examples which are not to be construed as limiting.

### EXAMPLE 1

### Exosome Isolation From Biological Samples Using a PEG-Based Method

Exosomes were isolated from various human and other mammalian biological samples as follows.

Blood samples were collected from healthy human subjects using red top serum collection tubes (e.g. BD, Ref #367812) and blue top plasma collection tubes containing sodium citrate (e.g. BD, Ref #369714) for serum and plasma isolations, respectively. For serum isolation, blood was allowed to clot for 1 hour at room temperature followed by centrifugation at 2,000×g for 15 min at 4° C. For plasma isolation, blood was spun down immediately after collection at 2,000×g for 15 min at 4° C. Plasma and serum was similarly collected from C57B1/6J mice and Sprague Dawley rats. Exosomes were then isolated from these samples, as well as from bovine whole milk (Natrel fine-filtered 3.25% milk). From this point onwards, all exosome sources were treated the same.

Serum, plasma and milk were spun at 2000×g for 15 min at 4° C. The supernatant from the first centrifugation was spun at 2000×g for 60 min at 4° C. to pellet debris. The supernatant was then spun at 15,000×g for 60 min at 4° C. The resulting supernatant was then filtered through a 45 μm filter (Millipore, cat. #SLHV033RS), followed by filtration through a 0.22 μm syringe filter (Millipore, cat. #SLGP0334B). The centrifugation and filtering steps have been determined to be optional steps. The filtered supernatant was then added to an equal volume of 16% PEG 6000 (Sigma, cat. #81253) and 500 mM NaCl in PBS (Bioshop, cat. #SOD002), mixed by inversion or gentle pipetting and incubated for 30 min at 4° C. The filtrate-PEG (8%) solution was then spun at 10,000×g for 10 min at 4° C. to pellet the exosomes. The supernatant was discarded and the pellet was solubilized in 600 uL of 0.5M trehalose (Sigma, cat. #T0167) in PBS by gentle pipetting or on a mechanical plate rocker for 30 min at 4° C. Exosomes were further purified by applying the exosome-containing solution to centrifugation between 15,000×g -150,000×g for 1 hr at 4° C. The resulting supernatant containing purified exosomes was then collected. A BCA assay (Pierce™) was used to determine exosome yield of between 5-10 mg of exosomal protein per 1 mL of serum used.

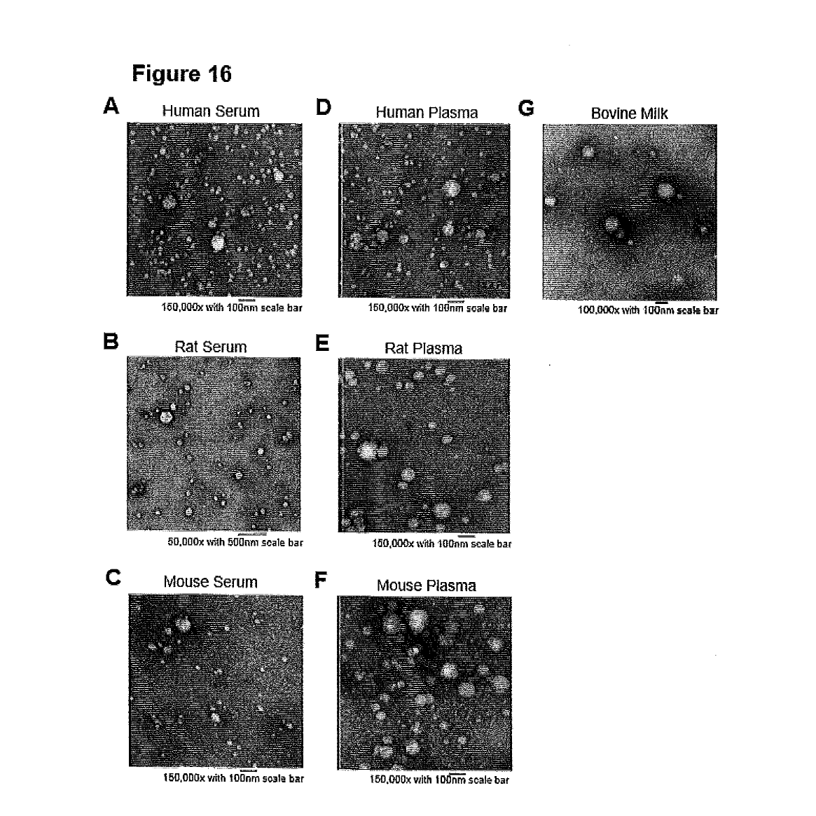

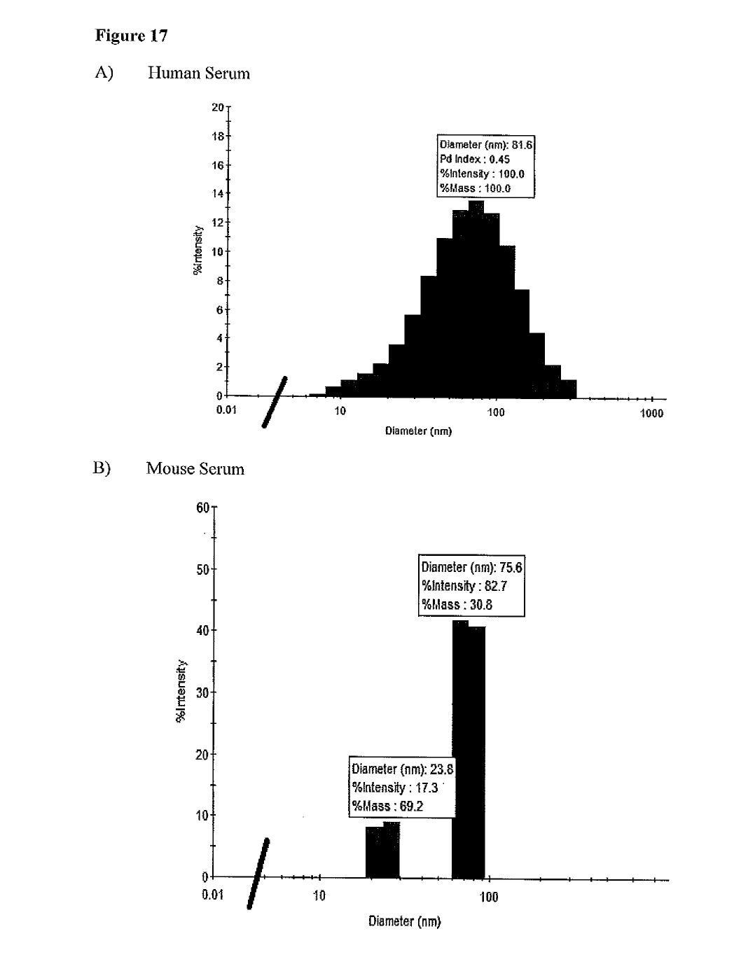

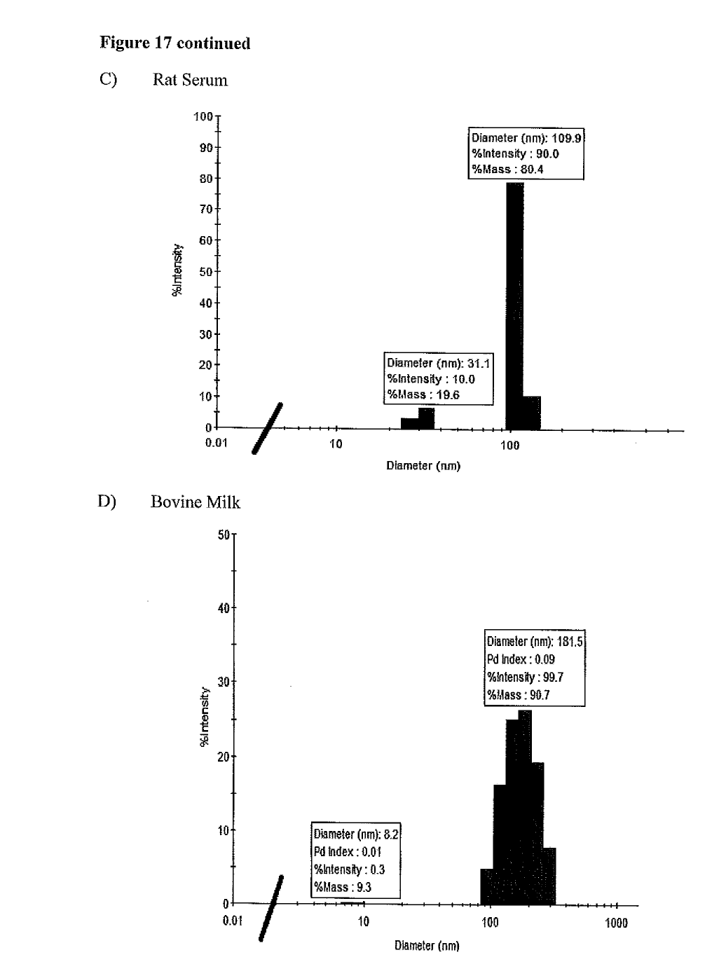

Transmission electron microscopy was performed on exosome solutions as shown in FIG. 16 confirming the isolation of exosomes in the size range of 20-140 nm in diameter from human serum (A, magnification 150,000× with 100 nm scale bar), rat serum (B, magnification 50,000× with 500 nm scale bar), mouse serum (C, magnification 150,000× with 100 nm scale bar), human plasma (D, magnification 150,000× with 100 nm scale bar), rat plasma (E, magnification 150,000× with 100 nm scale bar), mouse plasma (F, magnification 150,000× with 100 nm scale bar) and bovine milk (G, magnification 100,000× with 100 nm scale bar). The size distribution profile of exosomes isolated using the present PEG-based method from human serum (A), mouse serum (B), rat serum (C) and bovine milk (D) samples was then measured using a Beckman DelsaMax dynamic light scattering analyzer, showing that the majority of particles in these solutions were within the 20-140 nm size range with minimal contamination outside of this exosome size range (FIG. 17).

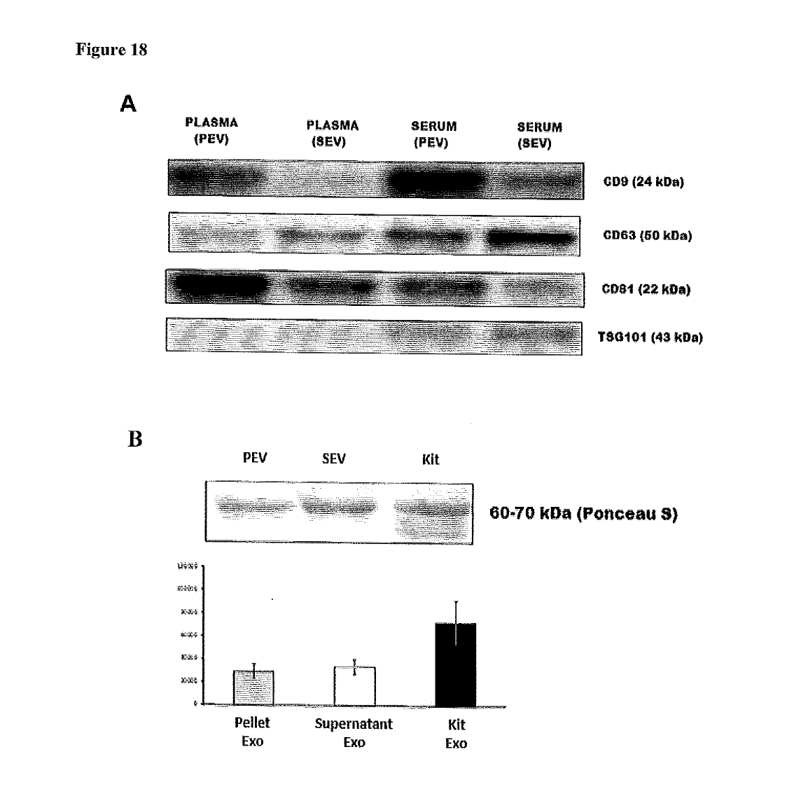

Exosomal purity was further exemplified by performing Western blots with the canonical exosome markers CD9, CD63, CD81 and TSG101. Both the supernatant and pellet fractions of exosome solutions isolated from mouse serum and plasma samples using the PEG-based isolation method (and a final ultracentrifugation step) demonstrated robust expression of these markers as shown in FIG. 18A, confirming the presence of exosomes. The purity of exosomes was also determined by performing a Ponceau S stain, a widely used indicator for the presence of protein bands during Western blotting. In comparison to mouse serum exosomes isolated with a commercially available kit, exosomes isolated using the PEG-based method have less Ponceau S staining in the size range of serum albumin which is one of the most abundant proteins in plasma (i.e. have less contamination by proteins such as albumin and other impurities) (see FIG. 18B). This indicates that exosome solutions resulting from the PEG-based method are of a higher purity than those isolated using a commercially available kit.

The loading capability of the isolated exosomes was then determined. The exosomes (100 μg of total exosomal protein) were loaded with 5 μg luciferase mRNA (Trilink, Cat. #L-6307) using cationic lipid-based transfection reagents (Lipofectamine® MessengerMAX™ Transfection Reagent, Life Technologies) according to manufacturer instructions. Exosomes (100 ug of total exosomal protein) were also loaded with 131 μg (100 μl) recombinant luciferase protein (Promega Corporation, Cat. #E1702) by combining the exosome solution and recombinant luciferase protein with an equal volume of 2% saponin in PBS solution, applying this mixture to an orbital rocker at room temperature for 30 minutes and then diluting the protein-loaded exosome solution in PBS. Loading of luciferase protein was confirmed upon measurement of luciferase protein activity in tissue lysates using standard methods.

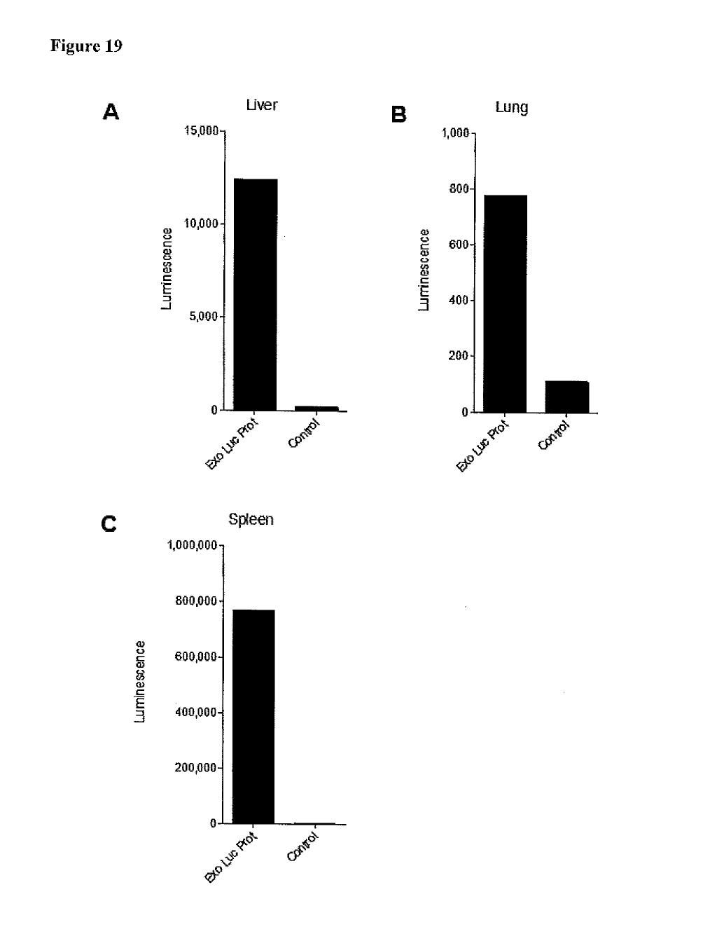

To demonstrate that exosomes can be used to deliver nucleic acids in vivo, wildtype mice were intravenously administered 100 μg of luciferase mRNA-loaded exosomes. Mouse liver, lung and spleen were then harvested 4 hours following injection. Luciferase activity was measured using a Luciferase Assay System (Cat. No. E1500; Promega Corporation) in tissue homogenates to quantify the amount of luciferase mRNA delivered to tissues. Control mice were intravenously administered 5 μg of luciferase mRNA in PBS. Mice treated with luciferase mRNA-loaded exosomes demonstrated luciferase activity in liver (A), lung (B) and spleen (C) tissues. Samples from control mice exhibited little or no luciferase activity (FIG. 19).

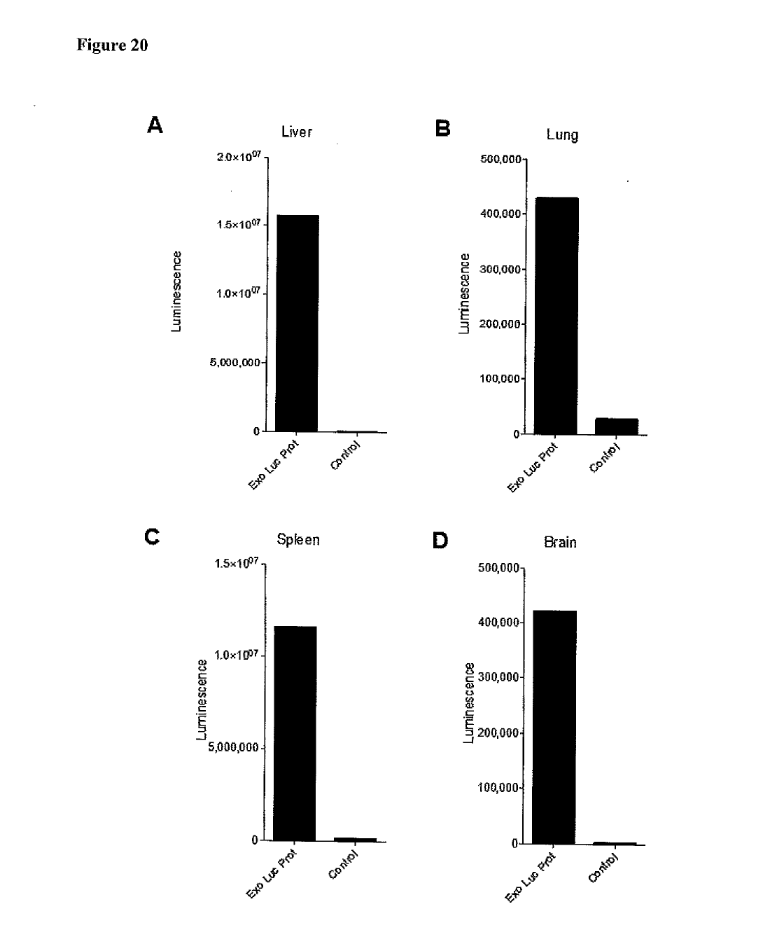

To similarly demonstrate that exosomes can be used to deliver exogenous proteins in vivo, 100 μg of protein loaded-exosomes were intravenously administered to wildtype mice. Mouse liver, lung, spleen and brain were then harvested 1 hour post injection. Luciferase activity was measured as described above. Control mice were intravenously administered 131 μg of luciferase protein in PBS. Mice treated with luciferase protein-loaded exosomes demonstrated luciferase activity in all tissues measured. Samples from control mice exhibited little or no luciferase activity (FIG. 20A-D).

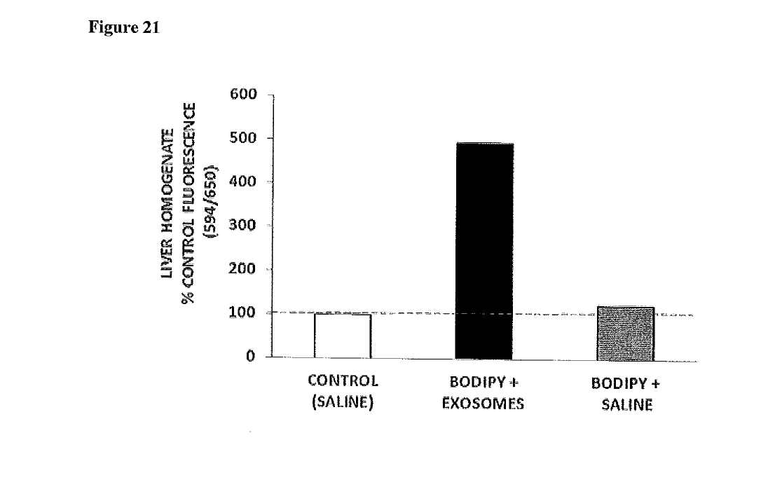

To further confirm that exosomes isolated using a PEG-based methodology can be effectively taken up in various tissues using an in vivo model system, isolated exosomes were labeled using BODIPY® TR ceramide fluorescent stain (Life Technologies). BODIPY® TR ceramide is a red fluorescent stain (absorption/emission maxima ˜589/617 nm), which is prepared from D-erythrosphingosine and has the same steriochemical conformation as natural biologically active sphingolipids. First, it was confirmed that BODIPY-labeled exosomes were taken up by human fibroblasts in vitro (not shown). Following this, 100 μg of labeled exosomes (suspended in PBS) were intravenously administered to C57/BL6 mice. Mouse liver was harvested 20 minutes following injection. Fluorescence was measured in tissue homogenates at 594/650 (Tecan Sapphire) and expressed relative to the two control groups comprised of either saline injection alone, or BODIPY and saline injected without exosomes. As demonstrated in FIG. 21, the amount of fluorescence was higher in the BODIPY-labeled exosome group, when compared to mice injected with BODIPY alone, confirming that exosomes themselves are taken up into tissue in an in vivo model.

### EXAMPLE 2

### Exosome Isolation From Cell Culture

Isolation of exosomes from cell culture (such as JawsII cells or Chinese hamster ovary (CHO) cells) was conducted using a similar procedure to that described in Example 1, including centrifugation of the sample, filtration, precipitation in PEG and solubilization in trehalose. In this case, however, the 0.22 μm filtrate was incubated with PEG at 4° C. overnight to enhance the precipitation of exosomes.

Exosome purity was similar to that achieved from the biological samples of Example 1. About 12-23 μg protein was obtained from about 1 mL of cell culture media.

### EXAMPLE 3

### Exosome Isolation From Human Biological Samples

Blood and urine samples were collected from healthy human subjects. For serum isolation, blood was allowed to clot for 1 hour at room temperature followed by spinning at 2,000× g for 15 min at 4° C. Similarly, urine samples were spun at 2,000×g for 15 min at 4° C. to remove any cellular debris. For plasma isolation, blood was spun down immediately after collection at 2,000×g for 15 min at 4° C. and treated with 5 ug of Proteinase K (20 mg/mL stock, Life Technologies) for 20 min at 37° C. From this point onwards, all samples (serum-1 mL, plasma-1 mL, and urine) are treated exactly the same.

The supernatant from the first centrifugation was spun at 2000×g for 60 min at 4° C. to further remove any contaminating non-adherent cells (optional). The supernatant was then spun at 14,000×g for 60 min at 4° C. (optional). The resultant supernatant was spun at 50,000×g for 60 min at 4° C. The resulting supernatant was then filtered through a 40 μm filter, followed by filtration through a 0.22 μm syringe filter (twice). The filtered supernatant was then carefully transferred into ultracentrifuge tubes and diluted with an equal amount of sterile PBS (pH 7.4, Life Technologies). This mixture was then subjected to ultracentrifugation at 110,000×-170,000×g for 2 hours at 4° C. using a fixed-angle rotor. The resulting pellet was then re-suspended in PBS and re-centrifuged at 110,000×-170,000×g for 2 hours at 4° C. (optional). The pellet was then resuspended carefully with 25 mL of sterile PBS (pH 7.4, Life Technologies) and gently added on top of 4 mL of 30%/70% Percoll™ gradient cushion (made with 0.22 μm filter sterilized water) or 30% Tris/Sucrose/sterile water cushion (300 g protease-free sucrose, 24 g Tris base, 500 ml sterile water, pH 7.4 and 0.22 μm filter sterilized) in an ultracentrifuge tube. This mixture was spun at 150,000×-170,000×g for 90 minutes at 4° C. With a syringe, the exosomal fraction (a distinct pellet at the gradient interface) was isolated carefully, diluted in 50 mL of sterile PBS (pH 7.4, Life Technologies) and spun for 90 minutes at 110,000×-170,000×g at 4° C. to obtain purified exosomes (this is optional when a sucrose gradient is used). The resulting exosomes was resuspended in sterile PBS or sterile 0.9% saline for downstream analyses (in vitro and in vivo). The purity of the exosomal fraction was confirmed by sizing, immuno-gold labeling/Western blotting using at least two independent exosome membrane markers, in this case, CD9 and CD63 were used.

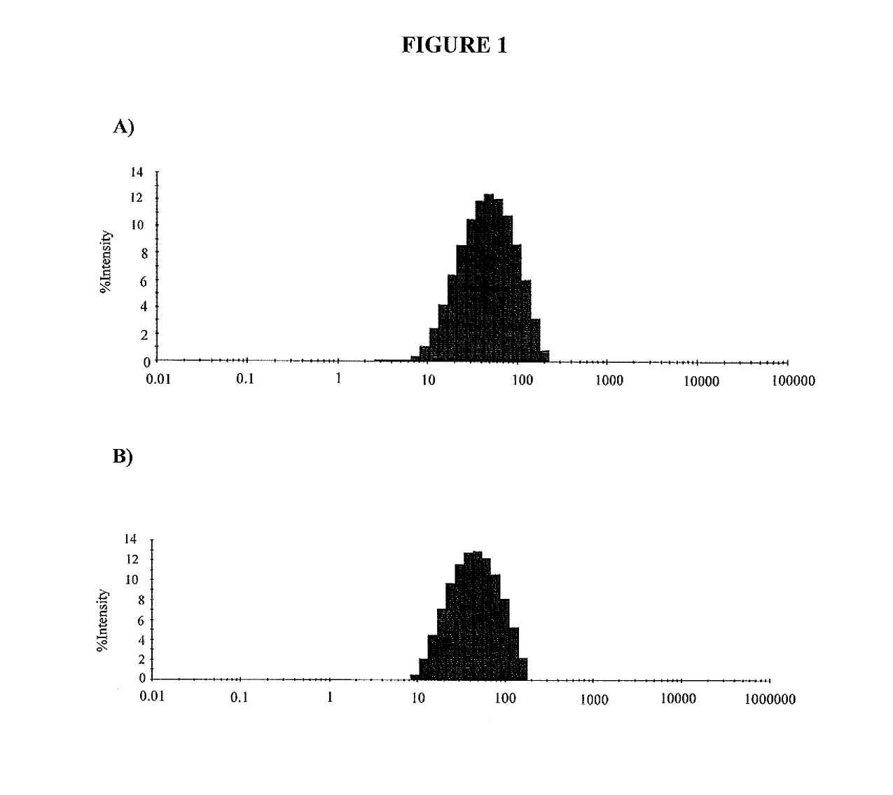

A BCA assay (Pierce™) was used to determine the yield of exosomes in each sample. The yield from serum, plasma and urine was determined to be in the range of 2-20 μg/μL, while the purity of the exosomal fraction was confirmed by qualitative immunogold-labeling, which indicated an average particle diameter of 90 nm, with minimal contamination outside of the 20-120 nm size range. The stability of the exosomes was also determined using a Beckman DelsaMax dynamic light scattering analyzer. The zeta potential of exosomes isolated from serum was determined to be −80.4 mV (see FIG. 1A).

### EXAMPLE 4

### Exosomes Isolated From Mice

Exosomes were isolated from 1 mL of serum obtained from C57B1/6J mice using the ultracentrifugation methodology as described in Example 1. An electron micrograph analysis of isolated exosomes was conducted to determine exosome size (magnification: 140,000×). Nanoparticle tracking analyses of isolated exosomes were visualized using a Beckman DelsaMax dynamic light scattering analyzer. Exosomes from mouse serum were determined to be about 90 nm in size on average (see FIG. 1B). Electron micrograph analyses of isolated exosomes immunogold-labeled with CD63 (exosomal membrane enriched marker) was conducted to confirm purity and integrity of serum exosomal fraction (magnification: 125,000×).

Total exosomal protein yield was determined to be 14.2 ug/uL by BCA assay. The stability of the isolated exosomes was determined using a Beckman DelsaMax dynamic light scattering analyzer. The zeta potential of exosomes isolated from serum was determined to be −78.1 mV.

### EXAMPLE 5

### Isolation From Dendritic Cells

Immature dendritic cells from human and mice are grown to 65-70% confluency in alpha minimum essential medium supplemented with ribonucleosides, deoxyribonucleosides, 4 mM L-glutamine, 1 mM sodium pyruvate, 5 ng/mL murine GM-CSF, and 20% fetal bovine serum. For conditioned media collection, cells were washed twice with sterile PBS (pH 7.4, Life Technologies) and the aforementioned media (with exosome-depleted fetal bovine serum) was added. Conditioned media from human and mouse immature dendritic cell culture was collected after 48 hours, The media (10 mL) was spun at 2,000×g for 15 min at 4° C. to remove any cellular debris. This is followed by an optional 2000×g spin for 60 min at 4° C. to further remove any contaminating non-adherent cells. The supernatant was then spun at 14,000×g for 60 min at 4° C. The resulting supernatant was spun at 50,000×g for 60 min at 4° C. The supernatant was then filtered through a 40 μm filter, followed by filtration through a 0.22 μm syringe filter (twice). The supernatant was then carefully transferred into ultracentrifuge tubes and diluted with an equal amount of sterile PBS (pH 7.4, Life Technologies). This mixture was then subjected to ultracentrifugation at 100,000×-170,000×g for 2 hours at 4° C. using a fixed-angle rotor. The resulting pellet was re-suspended in PBS and re-centrifuged at 100,000×-170,000×g for 2 hours at 4° C. The pellet was resuspended carefully with 25 mL of sterile PBS (pH 7.4, Life Technologies) and then added gently on top of 4 mL of 30%/70% Percoll™ gradient cushion (made with 0.22 μm filter sterilized water) in an ultracentrifuge tube. This mixture was spun at 100,000×-170,000×g for 90 minutes at 4° C. With a syringe, the exosomal pellet-containing fraction at the gradient interface was isolated carefully, diluted in 50 mL of sterile PBS (pH 7.4, Life Technologies), followed by a final spin for 90 minutes at 100,000×-170,000×g at 4° C. to obtain purified exosomes. The resulting exosomal pellet was resuspended in sterile PBS or sterile 0.9% saline for downstream use. Exosomal fraction purity was confirmed by sizing using a Beckman DelsaMax dynamic light scattering analyzer showing minimal contamination outside of the 40-120 nm size range, and by immuno-gold labeling/Western blotting using the exosome membrane markers, CD9, CD63, TSG101 and ALIX.

### EXAMPLE 6

### Isolation of Exosomes Front Plants

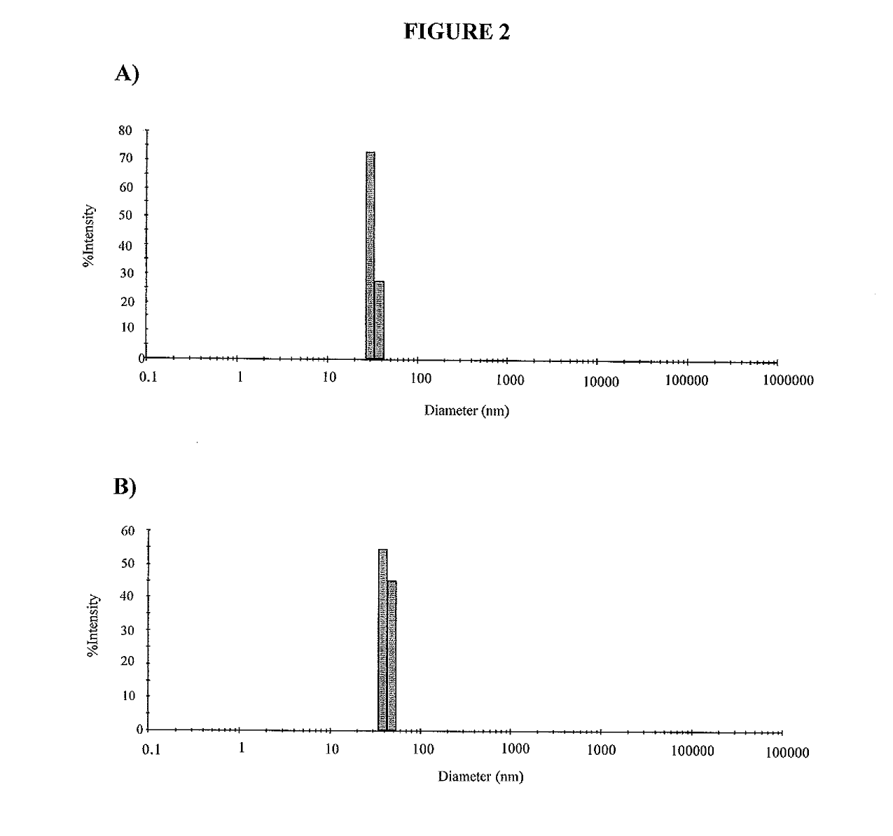

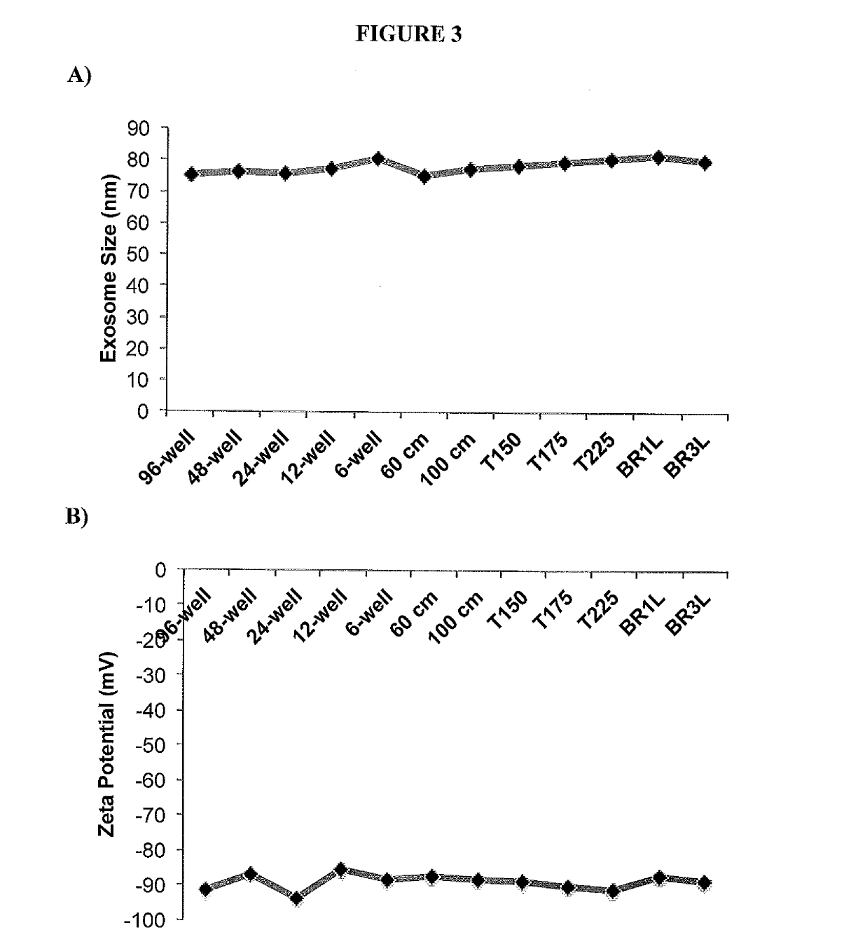

Kernels (from 4 corn cobs) and pomegranate seeds (from 4 pomegranates) were separately homogenized and homogenate was filtered using a 100 μm filter. The filtered homogenate was subjected to the exosome isolation protocol described in Example 1. As shown in FIG. 2 (A/B), exosomes were successfully isolated from these plants.

### EXAMPLE 7

### Exosome Production Scale Up

Exosomes have been successfully isolated from input samples of increasing size, for example, in well dishes from 96-, 48-, 24-, 12- to 6-well dishes; in well plates from 60- to 100-cm plates; and in flasks from T-150, T-175, to T-225 flasks (Corning). Isolated exosomes in each case exhibited similar quality and integrity, e.g. an average size of ˜78 nm (diameter) and zeta potential of about −87 mV (FIG. 3 A/B).

Exosome isolation from immature dendritic cells using 1 L and 3 L CELLine™ bioreactor flasks (Wheaton) was also conducted. The exosomes isolated from 1 L and 3 L bioreactors maintained an average size (diameter) and zeta potential similar to exosomes isolated on a smaller scale, as above.

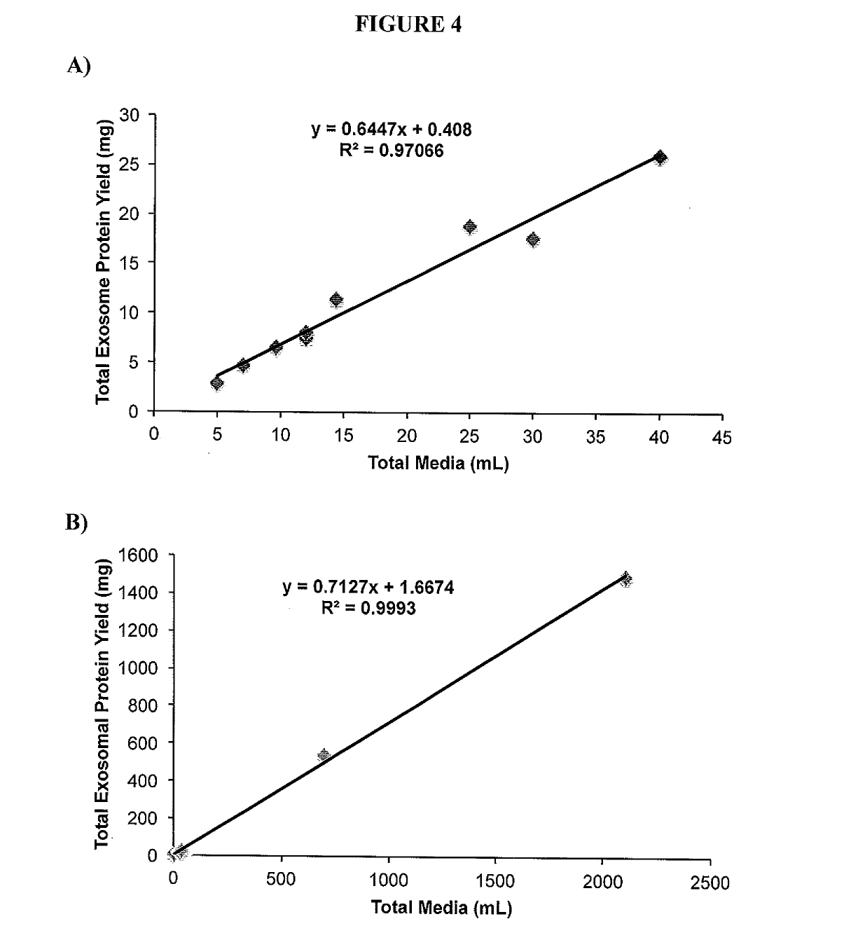

Thus, the present methods are readily scalable. The range of exosome protein yield achieved is linear from ˜4 mg (using 96-well plates) to ˜1400 mg (using a 3 L bioreactor) measured using BCA assay (Pierce) (FIG. 4 AIB).

### EXAMPLE 8

### Comparison of Exosome Isolation Techniques Using Commercially Available Kits

Commercially available exosome isolation kits from Life Technologies™, Cell Guidance System™, Norgen Biotek Corporation™, Qiagen™, Exigon™, and System Biosciences™ were used as per manufacturer's instructions to isolate exosomes from serum.

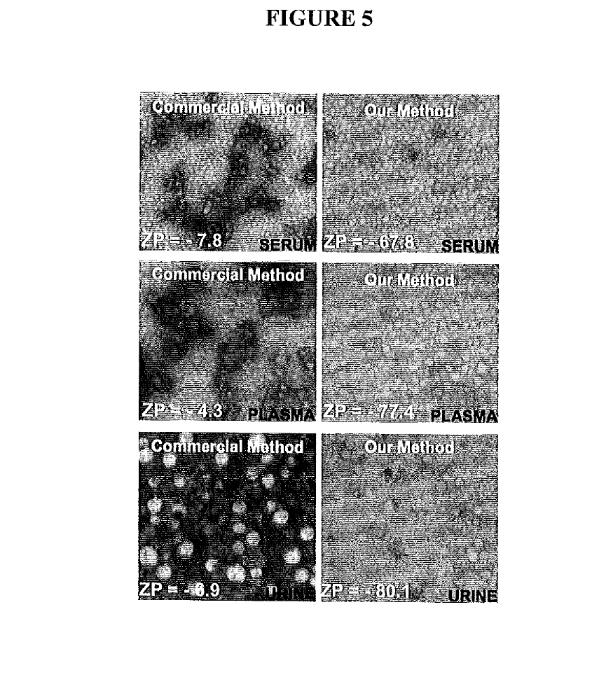

The results obtained using these kits were compared to the results obtained using the methods described in Examples 1-5. The quality of exosomes isolated using these kits was inferior to the quality of exosomes isolated using the methods of Examples 1-5. Specifically, as determined by electron microscopy analyses, the commercial kits yield a product containing contaminating debris and clumped microvesicles, while the methods of Examples 1-5 yielded circular exosomes having an average diameter of 90 nm that were not clumped (see FIGS. 5 and 16).

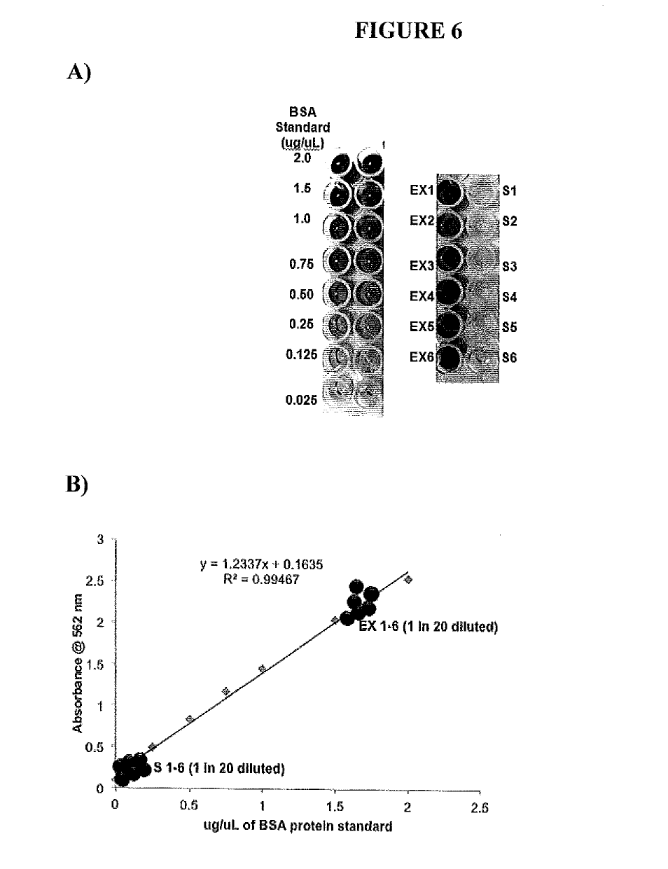

The quantity of exosomes isolated using isolating methods herein described was notably greater (10-25 μg/μL total protein as determined by BCA protein assay) than the protein quantity isolated using any of the commercial kits tested (0.1-0.5 μg/μL total protein as determined by BCA protein assay). Thus, the methods of Examples 3-5 yielded about 80-100× more exosomes as illustrated in FIG. 6 (EX1-EX6) in comparison to the protein yield of commercially available kits (S1-S6). In addition, the products isolated using commercial kits exhibited poor stability having a zeta potential of greater than −10 my (i.e. between −10 to 0 mV), and exhibited rapid coagulation/flocculation, in stark contrast to the stability of the exosomes isolated by the methods of Examples 3-5 which had a zeta potential of −80.4 mV (human) and −78.1 mV (mouse), which were circular and non-clumped. It was also noted that the products isolated using commercial kits were quite insoluble in physiological buffers as compared to the solubility in physiological buffer of the exosomes isolated using the present methods of Examples 3-5. The pellet obtained using commercially available kits could not be efficiently suspended in physiological buffer or in detergent-based buffers such as RIPA buffer or urea buffer.

### EXAMPLE 9

### Comparison of Exosome Isolation Techniques

Dr. Matthew J. A. Woods' laboratory has published two papers, Alvarez-Erviti et al., 2011. Nature Biotechnology, Vol. 29, 4:341, and El-Andaloussi et al., 2012. Nature Protocols, Vol 7, 12:2112, the relevant contents of each of which are incorporated herein, suggesting the use of exosomes for therapeutic delivery of siRNA in vivo. The protocols from Alvarez-Erviti et al. and El-Andaloussi et al. were used to obtain exosomes from spent media of immature dendritic cells. Briefly, these protocols involved centrifugation of spent media at 300 g for 10 min at 4° C. The resultant supernatant was then spun at 12,000 g for 30 min followed by a spin at 120,000 g for 70 min to pellet exosomes.

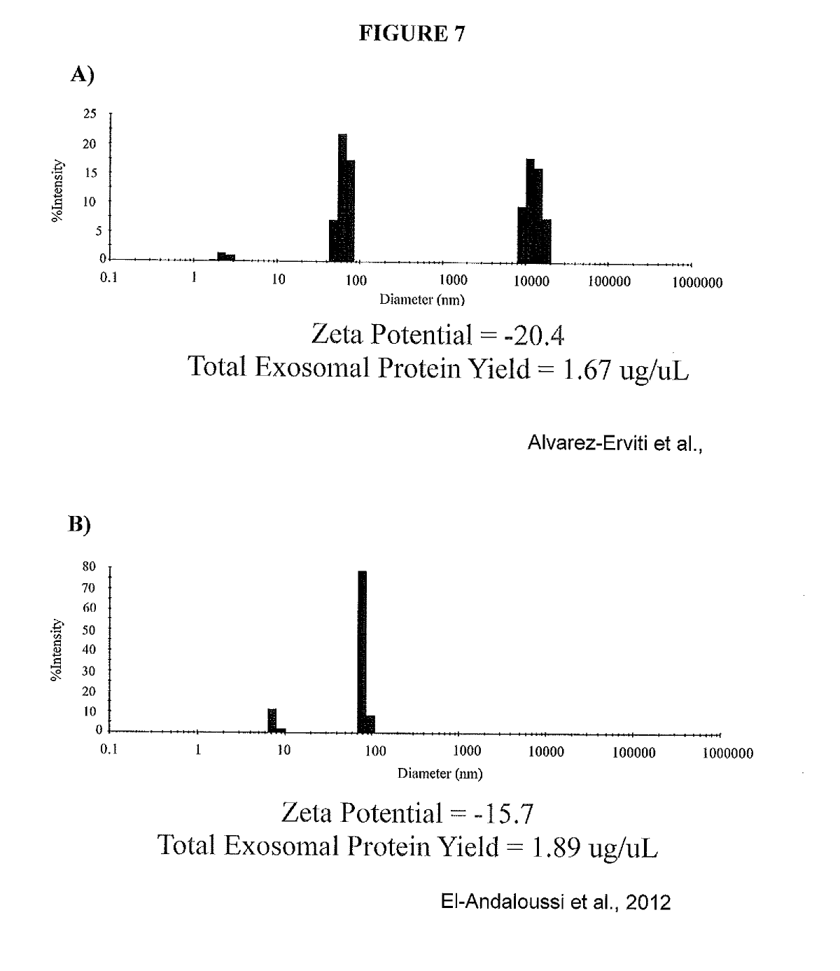

The exosome product attained using the protocol of Alvarez-Erviti et al. exhibited a protein yield 1.67 μg/μL, and included particles ranging in size from about 5 nm to greater than 1×10⁴ nm in diameter exhibiting a zeta potential of −20.4 mV (signifies incipient instability) as shown in FIG. 7A. The product using the protocol of El-Andaloussi et al. exhibited a protein yield 1.89 μg/μL, and included particles ranging in size from less than 10 nm to about 100 nm in diameter having a zeta potential of −15.7 mV as shown in FIG. 7B. This is in contrast to the product isolated by the present method (Examples 3-5), which exhibited a protein yield of 10-25 μg/μL total protein, exosomes having an average diameter of about 90 nm with no contaminating membrane fragments (e.g. less than 10 nm) or large microvesicles (greater than 1000 nm), and exosomes were determined to have a zeta potential in the excellent range, e.g. about −80.4 mV (human) and −78.1 mV (mouse).

To compare the utility of exosomes isolated according to El-Andaloussi et al., 2012, with exosomes isolated according to the present isolation method, loading of exosomes with siRNA as described in El-Andaloussi et al. was conducted. Specifically, C2C12 differentiated myotubes were treated separately with GAPDH siRNA packaged in El-Andaloussi et al. exosomes, and with GAPDH siRNA packaged in exosomes isolated according to the present isolation method. Exosomes were loaded with GADPH siRNA using electroporation at 400V and 125 μF, as described in El-Andaloussi et al. For positive and negative controls, myotubes were separately treated with GAPDH siRNA and scrambled siRNA (scRNA) combined with a transfection agent (Lipofectamine 2000).

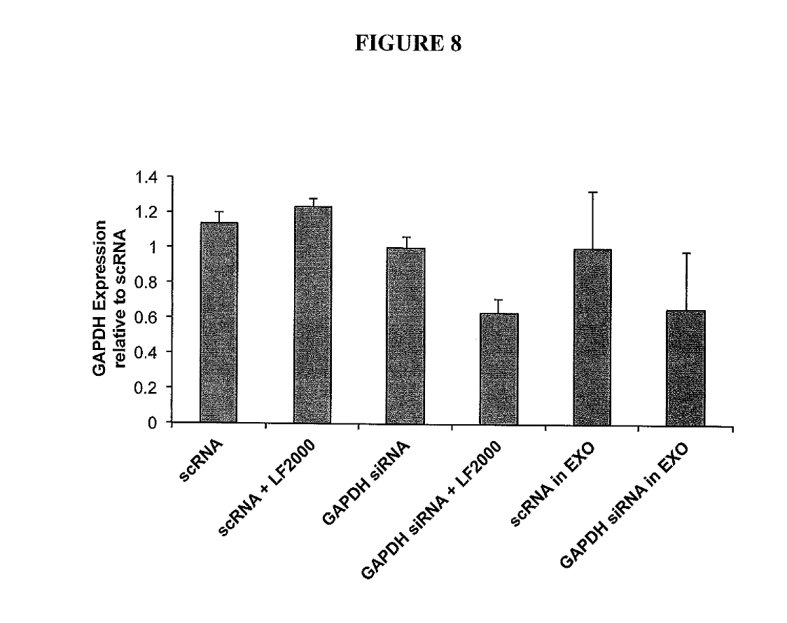

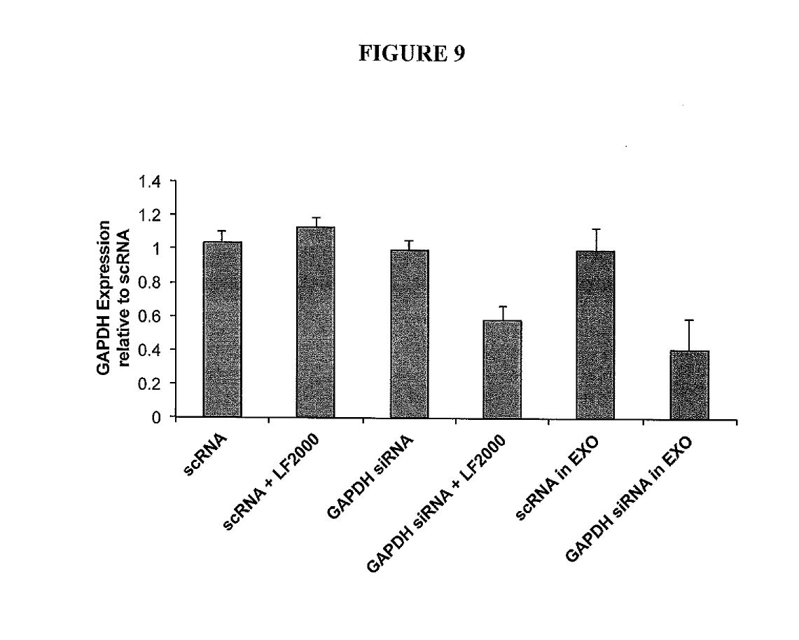

The El-Andaloussi et al. et al. exosomes packaged with siRNA exhibited about 35% silencing of GAPDH (FIG. 8) in three independent experiments. Exosomes isolated according to the present isolation method (Example 3) packaged with GADPH siRNA exhibited about 65% GAPDH silencing (FIG. 9). Thus, exosomes isolated according to the present method exhibited a greater capacity for cargo uptake in view of their significantly improved performance with respect to gene silencing.

The capacity of El-Andaloussi et al. exosomes to load or package other types of cargo was also determined and compared to the loading capacity of exosomes isolated according to the present isolation method. Loading of exosomes was conducted by electroporation as described above. Exosomes were loaded with various cargo types, including siRNA (GADPH, Sigma-Aldrich), miRNA (mmu-miR-23a, Accession #MI0000571), mRNA (GAA, NCBI Reference Sequence: NM_001159324.1) and peptide (rGAA; NCBI Reference Sequence: NP_001152796.1).

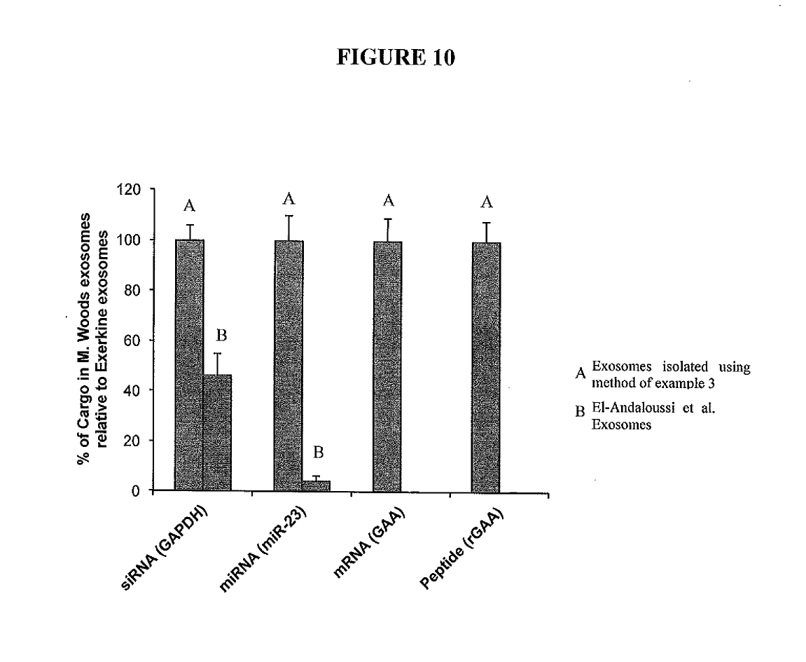

As shown in FIG. 10, El-Andaloussi et al. exosomes exhibited a limited capacity for cargo loading. While these exosomes exhibited some loading of siRNA, loading with miRNA, mRNA and peptide was insignificant or non-existent. In comparison, exosomes isolated according to the present methods exhibited 100% loading of each of siRNA (2.22-fold greater loading for siRNA compared to exosomes isolated using El-Andaloussi et al. protocol), miRNA (25-fold greater compared to exosomes isolated using El-Andaloussi et al. protocol), mRNA and peptide (an infinite-fold greater loading given that absolutely no mRNA or peptides could be loaded using El-Andaloussi et al. exosomes).

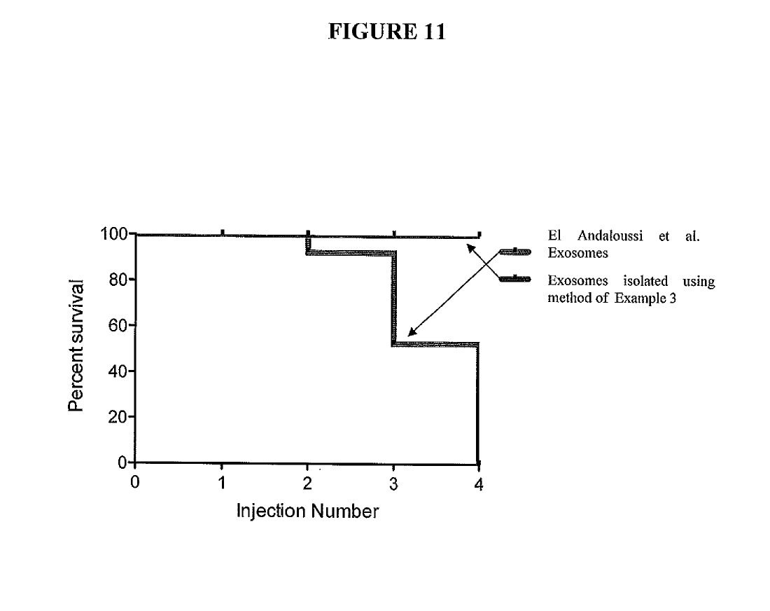

Lastly, the safety of El-Andaloussi et al. exosomes was compared to exosomes isolated according to the present isolation method. To assess safety, C57B116J mice (6 per group) were given four intravenous injections (one injection per week) with 10 ug (exosome total protein) of empty xenogenic exosomes (re-suspended in 0.9% sterile saline) prepared from human immature dendritic cells. These mice were not given an antihistamine (e.g. Benadryl) prior to or during exosome treatments. All mice injected with exosome product prepared according to El-Andaloussi et al. died by the fourth injection, while all mice injected with exosomes isolated according to the method of Ex. 3 exhibited no mortality or adverse reaction (see FIG. 11).

### EXAMPLE 10

### Loading of Modified RNA Into Exosomes and Delivery

The capacity of exosomes isolated according to the present isolation method (Ex. 3) to load and deliver modified RNA, was compared to the delivery of naked modified RNA. Exosomes (20 ug of total exosomal protein) were loaded with 500 ng of unmodified (physiological) mRNA-Vegfa and 500 ng of modRNA-Vegfa (synthesis of which is described in Zangi et al., 2013 Nature Biotechnology, 31, 898-907, the relevant contents of which are incorporated herein). Loading was accomplished using a cation-based transfection reagent as previously described.

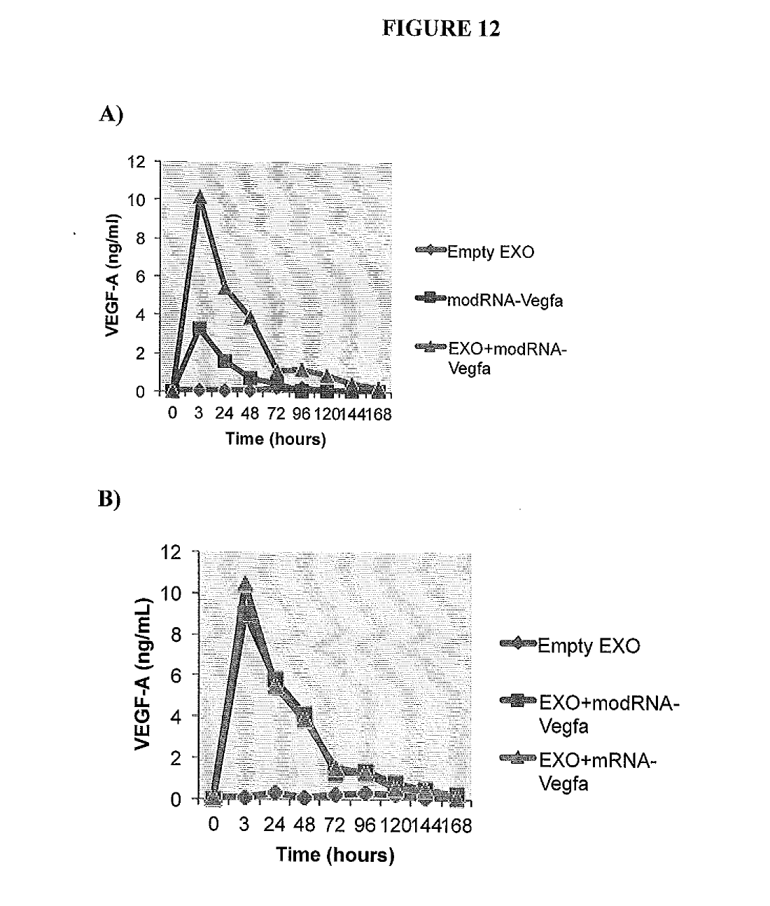

Primary cardiomyocytes were treated with either empty exosomes, modRNA-Vegfa, modRNA-Vegfa-loaded exosomes or mRNA-Vegfa-loaded exosomes. Cells treated with modRNA-Vegfa alone were subjected to Lipofectamine 2000 for efficient transfection by the cells. VEGF-A protein production over time (0-180 hours) was used as a readout. Treated cells showed a steady production of VEGF-A protein that peaked at ˜20 hours and came back to baseline ˜144 hours; however, the total amount of VEGF-A produced in cells treated with either mRNA-Vegfa exosomes or modRNA-Vegfa exosomes (about 10 ng/ml) was about 3× the amount produced by cells treated with modRNA-Vegfa alone (about 3.2 ng/ml) (see FIG. 12 A/B). Thus, unmodified and modified RNA, e.g. mRNA-Vegfa and modRNA-Vegfa is efficiently loaded into exosomes and delivered to cells without the use of exogenous transfection reagents.

Additionally, it is evident that when using exosomes as therapeutic delivery vehicles, no modifications to native mRNA (modRNA) are needed nor do these modifications provide any therapeutic advantage when compared to exosomes packaged with native mRNA (FIG. 12 B).

### EXAMPLE 11

### Biodistribution of Exosomes

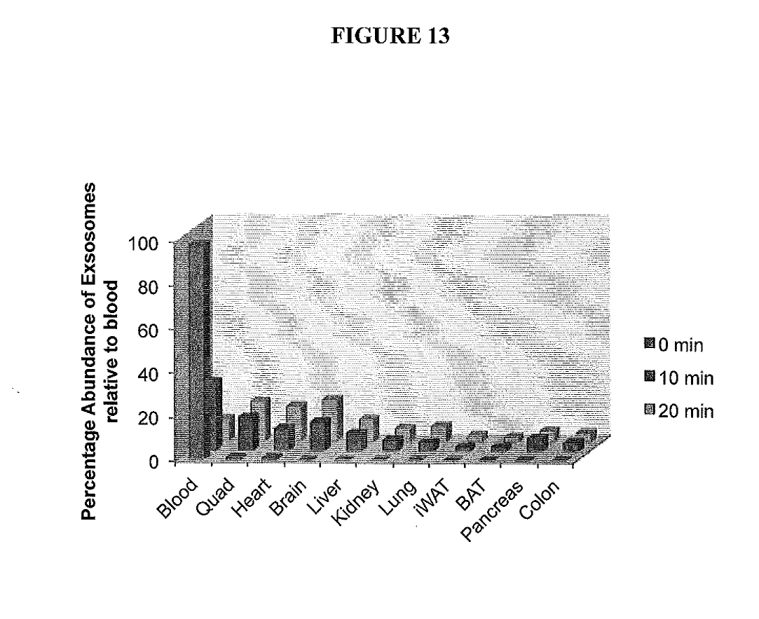

To confirm that exosomes isolated according to the present isolation method (e.g. Ex. 3) can be effectively taken up by various tissues in an in vivo model system, isolated exosomes were labeled using BODIPY® TE ceramide fluorescent stain (Life Technologies). BODIPY® TR ceramide is a red fluorescent stain (absorption/emission maxima ˜589/617 nm), which is prepared from D-erythrosphingosine and has the same steriochemical conformation as natural biologically active sphingolipids. 100 ug of total labeled exosomes (suspended in sterile 0.9% saline) were intravenously administered to mice. Mice tissues/organs (quadriceps, heart, brain, liver, kidney, lung, inguinal white adipose tissue, brown adipose tissue, pancreas, and colon) and blood were then harvested immediately (0 min), at 10 min following injection, and at 20 minutes following injection (4 mice per group). Fluorescence was measured in serum and tissue homogenates and expressed relative to blood (plasma) to quantify the amount of labeled exosomes in various tissues/organs. At time 0 min, the majority of the fluorescence was observed in blood, and over time (10 min and 20 min), an increase of fluorescence in various tissues/organs occurred as non-specific global biodistribution of labeled exosomes (FIG. 13).

### EXAMPLE 12

### Exosome Bioactivity Studies In Vitro

The bioactivity of exosomes isolated as described herein was compared with the bioactivity of exosomes isolated using a commercially available kit, using a human primary dermal fibroblast proliferation study. Human primary dermal fibroblasts were isolated from skin biopsies of healthy human subjects using standard procedure. Exosomes were isolated from serum from sedentary (SED) and athletic (END) individuals as described herein (e.g. Example 3) and using a commercially available exosome isolation kit. The dermal fibroblasts were separately treated with equal amounts of isolated exosomes (100 ng/μL total exosomal protein) for 5 days in culture (n=3/treatment).

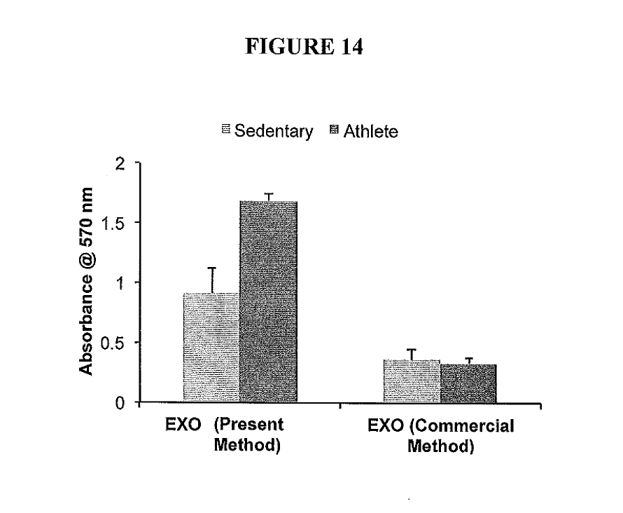

Vybrant® MTT cell proliferation assay (Life Technologies) was carried out to investigate cellular proliferation following exosome treatment. As shown in FIG. 14, SED and END exosomes isolated using the present method exhibit an enhanced proliferative effect on human dermal fibroblasts as compared to the effect of SED and END exosomes isolated from the same sample using a commercial method. Note that serum samples used for this comparison were collected from the same athlete and sedentary individual at the same time to prevent any effect of physiological variability (such as using different subjects or sampling times). Data were analyzed using an unpaired I-test and are presented as mean±SEM.*P<0.05 for Sedentary vs. Athlete groups.

While not wishing to be limited to a particular explanation, the lack of bioactivity in exosomes isolated from commercially available kits may be due to insufficient purification of the exosomes in combination with mechanical shear of the exosomes in a hydrophobic environment and “clumping” of the exosomes.

### EXAMPLE 13

### Bioactivity of Isolated Exosomes In Vivo

Exosomes isolated according to the present methods (e.g. Example 3) were found to be biologically active in an in vivo environment. Male C57B1/6J mice, bred in an institutional central animal facility (McMaster University), were housed in micro-isolator cages in a temperature- and humidity-controlled room and maintained on a 12-h light-dark cycle with food and water ad libitum. At 3 months of age, mice (N=150/group) were randomly assigned to either sedentary (SED—housed in a wheel cage, wheel locked to prevent exercising) or exercise (END—treadmill training: 15 m/min for 60 min, 5×/week for 2 months using Eco 3/6 treadmill; Columbus Instruments) groups ensuring that body mass was similar between groups. Before exosome harvest from the mice, an endurance stress test was carried out as previously described (Safdar et al., 2011, PNAS, 108(10):4135-40) to determine that mice in the END group had a higher aerobic capacity than SED mice.

Exosomes were isolated from blood of both the SED and END mice. Isolated exosomes were re-suspended in sterile 0.9% saline to a concentration of 1 μg/μL total exosomal protein. Exosome solution, either exosomes from SED mice or exosomes from END mice, was intravenously administered (100-150 μL exosome saline solution) 5×/week with 1 to 1 donor-recipient ratio to an independent cohort of SED mice. After 6 weeks of treatment, mice from both groups (SED mice getting SED exosomes and SED mice getting END exosomes) were housed in voluntary activity cages (Columbus Instruments) for 24 hours to measure their voluntary exercise capacity. These mice were then subjected to an endurance stress test. A separate cohort of C57B1/6J sedentary (SED) or endurance trained (END; trained in voluntary wheel running cages for 10 weeks) mice were subjected to an endurance stress test as negative and positive control of endurance exercise adaptations, respectively. Data were analyzed using an unpaired t-test and are presented as mean±SEM.*P<0.05 for SED vs. END groups; §P<0.05 for SED+SED EXO vs. SED+END EXO groups.

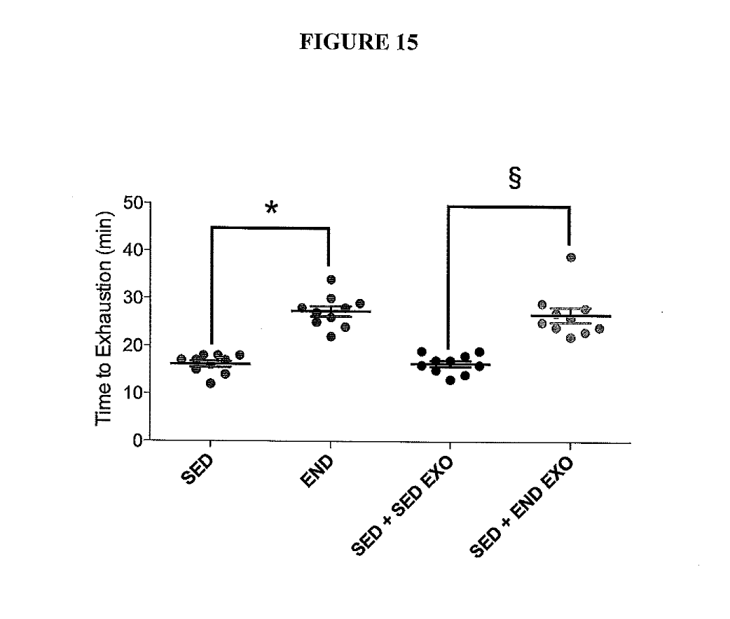

An increase in basal voluntary activity and maximum endurance capacity of sedentary mice was observed when they were given END exosomes in contrast to the effect of SED exosomes given to SED mice (FIG. 15). The basal voluntary activity and maximum endurance capacity of sedentary mice that were given END exosomes for 6 weeks while maintaining their ‘sedentary status’ was comparable to that of mice that were trained in voluntary wheel cages for 6 weeks.

Relevant portions of references referred to herein are incorporated by reference.

1. A method of isolating exosomes from a biological sample comprising the steps of:
i) optionally exposing the biological sample to a first centrifugation to remove cellular debris greater than about 7-10 microns in size from the sample and obtaining the supernatant following centrifugation;
ii) optionally subjecting the supernatant from step i) to centrifugation to remove microvesicles and apoptotic bodies therefrom;
iii) optionally microfiltering the supernatant from step ii) and collecting the microfiltered supernatant;
iv) combining the microfiltered supernatant from step iii) with a polyethylene glycol (PEG) solution to precipitate the exosomes and subjecting the solution to at least one round of centrifugation to obtain an exosome pellet; and
v) re-suspending the exosome pellet from step iv) in a trehalose solution and conducting an optional centrifugation step to remove vesicles having a diameter of greater than 140 nm from the solution.

2. The method of claim 1, wherein the PEG solution comprises PEG chain lengths having an average molecular weight of between about 400 to 20,000 daltons and a concentration of about 10-20% PEG.

3. The method of claim 1, wherein the trehalose solution has a concentration of about 10 mM to 1,000 mM.

4. The method of claim 1, wherein step ii) comprises centrifugation at a speed of between 40,000-60,000×g for 30-90 minutes.

5. The method of claim 4, wherein the centrifugation is conducted at a speed of 50,000 for 1 hour.

6. The method of claim 1, wherein step ii) comprises a second centrifugation of the supernatant at a speed of between 12,000-15,000×g for 30-90 minutes, and the resulting supernatant is subjected to a third centrifugation at a speed of between 40,000-60,000×g for 30-90 minutes.

7. The method of claim 6, wherein the second centrifugation is conducted at a speed of 14,000×g for 1 hour and the third centrifugation is conducted at a speed of 50,000 for 1 hour.

8. The method of claim 1, wherein the first centrifugation is conducted at a speed of between 1000 to 4000×g for 10 to 60 minutes, and wherein the first centrifugation is optionally repeated.

9. The method of claim 1, wherein the supernatant of step iii) is microfiltered at least once using a 0.2-10 micron filter, and preferably a 0.22 micron filter.

10. An exosome pellet or physiological solution comprising exosomes essentially free from particles having a diameter less than 20 nm or greater than 140 nm.

11. The pellet or solution of claim 10, wherein the exosomes are loaded with an exogenous cargo.

12. The pellet or solution of claim 11, wherein the exogenous cargo is selected from the group consisting of DNA, siRNA, mRNA, tRNA, aptamers, miRNA, peptides, proteins, ribozymes, carbohydrates, therapeutic compounds, small molecules, and polymers.

13. The pellet or solution of claim 11, wherein the cargo comprises secondary structure or is greater than 20 base pairs in size.

14. The pellet or solution of claim 11, wherein the cargo is mRNA.

15. The pellet or solution of claim 11, wherein the cargo is a protein.

16. A kit for use in a method of isolating exosomes from a biological sample as defined in claim 1, comprising a polyethylene glycol solution to precipitate the exosomes and a trehalose solution to reduce exosome aggregation.

17. A method of in vivo delivery of exogenous cargo to a mammal comprising the step of administering to the mammal an physiologically acceptable exosome solution which is essentially free from particles having a diameter less than 20 nm or greater than 140 nm, wherein the exosomes are loaded with the exogenous cargo.

18. The method of claim 17, wherein the cargo comprises secondary structure or is greater than 20 base pairs in size.

19. The method of claim 18, wherein the cargo is a protein or mRNA.

20. The method of claim 17, wherein the cargo is delivered to muscle, heart, brain, liver, kidney, lung, inguinal white adipose tissue, brown adipose tissue, pancreas or colon.
